(19) United States
(12) Patent Application Publication
(10) Pub. No.: US 2026/0170260 A1
(43) Pub. Date: Jun. 18, 2026

(54) INFORMATION PROCESSING APPARATUS, PROCESSING METHOD OF INFORMATION PROCESSING APPARATUS, AND STORAGE MEDIUM STORING PROGRAM

(71) Applicant: CANON KABUSHIKI KAISHA, TOKYO (JP)

(72) Inventor: TOMOHIRO KAWAMOTO, TOKYO (JP)

(21) Appl. No.: 19/402,808

(22) Filed: Nov. 26, 2025

(30) Foreign Application Priority Data
Dec. 18, 2024 (JP) 2024-221769

(51) Int. Cl. G06F 40/35; G16H 10/60

(52) U.S. Cl. CPC G06F 40/35; G16H 10/60

(57) ABSTRACT

An information processing apparatus performs a dialogue with a client, estimation unit estimates a mental condition of the client by analyzing the dialogue with the client, where, based on the estimated mental condition, a dialogue with the client is performed to change the dialogue with the client to a dialogue between the client and a dialogue partner.

### BACKGROUND

### Field of the Technology

The present disclosure relates to an information processing apparatus, a processing method of the information processing apparatus, and a storage medium storing a program.

### Description of the Related Art

There are many persons who have mental health problems and persons who are at risk of developing mental problems. Since the number of counselors to provide assistance is currently not enough, it is difficult to provide counseling to those currently with a health problem and those who are at risk of developing a mental health problem. Thus, mental health technology (digital tools or applications for assisting mental health) has begun to appear.

Japanese Patent Laid-open No. 2020-155041 describes an information processing apparatus that receives emotion information regarding a user's emotions from the user, and, based on the emotion information and input environment information, presents a reply statement in response to an input statement from the user.

Japanese Patent Laid-open No. 2019-185586 describes a stress management assistance system including a humanoid robot provided with a video sensor having a function of reading facial expressions, and a voice sensor having a function of hearing voices, and artificial intelligence. The artificial intelligence has a function of interactively performing stress coping, cognitive behavioral therapy, or assertiveness.

The information processing apparatus described in Japanese Patent Laid-open No. 2020-155041 only presents the reply statement in response to the input statement from the user, and does not describe a countermeasure of a method of how to deal with a case where the symptom of the person with the mental problem is not improved.

The stress management assistance system described in Japanese Patent Laid-open No. 2019-185586 is a system only for interactively performing stress coping, cognitive behavioral therapy, or assertion, and does not describe a method of how to deal with a case where the symptom of the person with the mental problem is not improved by the interactive format of the artificial intelligence.

### SUMMARY

The present disclosure is directed to performing a dialogue between a client and a dialogue partner (counselor) according to a mental condition of a client.

According to an aspect of the present disclosure, an information processing apparatus includes at least one memory storing a program and at least one processor, that when executing the program, is caused to perform a first dialogue with a client, and to estimate a mental condition of the client by analyzing the first dialogue, wherein, based on the estimated mental condition, the perform a dialogue with the client to change the first dialogue to a second dialogue between the client and a dialogue partner.

Features of the present disclosure will become apparent from the following description of embodiments with reference to the attached drawings. The following description of embodiments is described by way of example.

### BRIEF DESCRIPTION OF THE DRAWINGS

FIG. 1 is a block diagram illustrating an example of a configuration of an information processing system according to a first embodiment.

FIG. 2 is a block diagram illustrating an example of a common hardware configuration of a user's information terminal and a counselor's information terminal.

FIG. 3 is a block diagram illustrating an example of a hardware configuration of a server.

FIG. 4 is a block diagram illustrating an example of a functional configuration of the server.

FIG. 5 is a diagram illustrating an example of a screen of a dialogue displayed on a display unit of the user's information terminal.

FIG. 6 is a flowchart illustrating a processing method performed by the server.

FIG. 7 is a diagram illustrating an example of a screen of a dialogue displayed on the display unit of the user's information terminal.

FIG. 8 is a diagram illustrating an example of a screen of a dialogue displayed on the display unit of the user's information terminal.

FIG. 9 is a diagram illustrating an example of a screen of a dialogue displayed on the display unit of the counselor's information terminal.

FIG. 10 is a flowchart illustrating a processing method performed by the server.

FIG. 11 is a diagram illustrating an example of a screen of a dialogue displayed on the display unit of the user's information terminal.

FIG. 12 is a diagram illustrating an example of a screen of a dialogue displayed on the display unit of the counselor's information terminal.

FIG. 13 is a diagram illustrating an example of a medical record.

FIG. 14 is a diagram illustrating an example of another medical record.

FIG. 15 is a diagram illustrating follow-up processing performed by the server.

FIG. 16 is a flowchart illustrating a processing method performed by a server according to a second embodiment.

FIGS. 17A and 17B are diagrams illustrating processing performed by an identification dialogue model.

### DESCRIPTION OF THE EMBODIMENTS

### First Embodiment

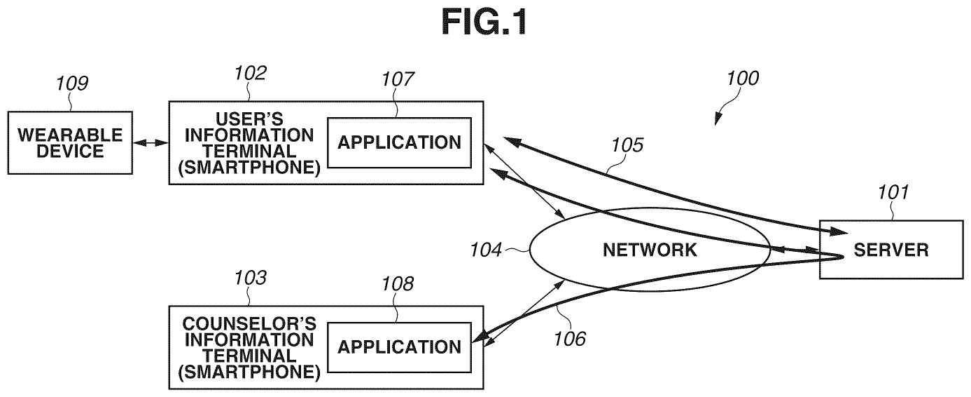

FIG. 1 is a block diagram illustrating an example of a configuration of an information processing system 100 according to a first embodiment. The information processing system 100 includes a server 101, a user's information terminal 102, a counselor's information terminal 103, a network 104, and a wearable device 109.

The user's information terminal 102 and the counselor's information terminal 103 are, for example, smartphones. The server 101 is an example of an information processing apparatus, and is, for example, a computer.

The user's information terminal 102 and the server 101 communicate with each other via the network 104. The counselor's information terminal 103 and the server 101 communicate with each other via the network 104. The user's information terminal 102 and the counselor's information terminal 103 communicate with each other via the network 104 and the server 101.

The user's information terminal 102 is an information terminal used by a user. The user is, for example, a patient suspected of having a mental disorder, and the user can request counseling about the user's mental disorder using a user counseling application 107 in the user's information terminal 102. The user is a client of the counseling. The user counseling application 107 is a computer program used by the user for accessing counseling services supplied by the server 101.

The counselor's information terminal 103 is an information terminal used by the counselor. The counselor is a counselor for mental disorders, and is, for example, a healthcare professional such as a “clinical psychologist” or a “certified psychologist” who performs psychological counseling (psychological therapy). The counselor can accept counseling about a mental disorder using a counselor counseling application 108 in the counselor's information terminal 103. The counselor is a user's dialogue partner of the counseling. The counselor counseling application 108 is a computer program used by the counselor for the counseling services provided by the server 101.

The server 101 provides counseling services to the user's information terminal 102 and the counselor's information terminal 103 using a dialogue model.

The wearable device 109 and the user's information terminal 102 communicate with each other. The wearable device 109 is, for example, an “Apple Watch®”, and is worn on the user's body. The wearable device 109 detects ambient light, acceleration, a gyroscope, heartbeat, atmospheric pressure, a compass, and blood oxygen level, and transmits the detected results to the server 101 via the user's information terminal 102.

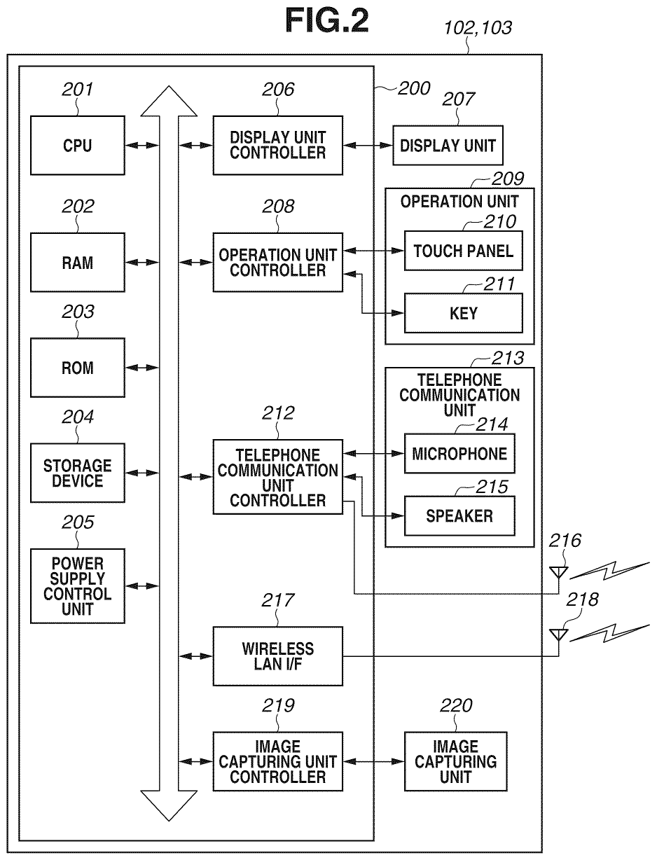

FIG. 2 is a block diagram illustrating an example of a common hardware configuration of the user's information terminal 102 and the counselor's information terminal 103 illustrated in FIG. 1. The user's information terminal 102 and the counselor's information terminal 103 are, for example, smartphones.

The user's information terminal 102 and the counselor's information terminal 103 include a controller unit 200, a display unit 207, an operation unit 209, a telephone communication unit 213, a voice communication antenna 216, a wireless local area network (LAN) antenna 218, and an image capturing unit 220.

The controller unit 200 includes a central processing unit (CPU) 201, a random-access memory (RAM) 202, a read-only memory (ROM) 203, a storage device 204, a power supply control unit 205, a display unit controller 206, an operation unit controller 208, a telephone communication unit controller 212, a wireless LAN interface (I/F) 217, and an image capturing unit controller 219. The operation unit 209 includes a touch panel 210 and a key 211. The telephone communication unit 213 includes a microphone 214 and a speaker 215.

The CPU 201 is a central processing unit that controls the user's information terminal 102 or the counselor's information terminal 103.

The RAM 202 is a work memory for the CPU 201 to operate with, and is used for loading various programs therein and storing calculation processing results.

The ROM 203 is a memory for storing a start-up program of the CPU 201 and various kinds of setting information.

The storage device 204 is a nonvolatile memory for storing large size programs and data, and is a flash memory or a hard disk drive (HDD) in the controller unit 200. The storage device 204 in FIG. 1 stores the user counseling application 107 or the counselor counseling application 108.

The power supply control unit 205 includes a battery and a battery charge module, and controls electric power required for the user's information terminal 102 or the counselor's information terminal 103 to operate.

The display unit controller 206 transmits to the display unit 207, based on a communication protocol of the display unit 207, characters for the dialogue or an image for a video communication for the counseling.

The operation unit controller 208 receives an input from the touch panel 210 and the key 211, and converts the received input into data understandable by the CPU 201. The touch panel 210 and the display unit 207 are integrally configured, and the touch panel 210 can detect a touch operation performed on the surface of the display unit 207.

The telephone communication unit controller 212 performs processing of converting voice signals received from the microphone 214 into radio waves for transmitting external to the information terminal 102 or the counselor's information terminal 103 by outputting it to the voice communication antenna 216. In addition, the telephone communication unit controller 212 also converts radio waves received by the voice communication antenna 216 external from the information terminal 102 or the counselor's information terminal 103 into voice data and outputting it to the speaker 215.

The image capturing unit 220 captures an image of an object with a camera to generate image data. The image capturing unit controller 219 controls the image capturing unit 220.

The wireless LAN I/F 217 transmits and receives data via the wireless LAN antenna 218 to and from the network 104 illustrated in FIG. 1. The wireless LAN I/F 217 of the user's information terminal 102 transmits and receives data via the wireless LAN antenna 218 to and from the wearable device 109 illustrated in FIG. 1.

The CPU 201 can perform voice communication using the telephone communication unit 213 and the wireless LAN I/F 217 by executing the user counseling application 107 or the counselor counseling application 108. The voice communication is performed between the user's information terminal 102 and the counselor's information terminal 103 via the server 101.

The CPU 201 can perform video communication using the telephone communication unit 213, the image capturing unit 220, and the wireless LAN I/F 217, by executing the user counseling application 107 or the counselor counseling application 108. The video communication is performed between the user's information terminal 102 and the counselor's information terminal 103 via the server 101.

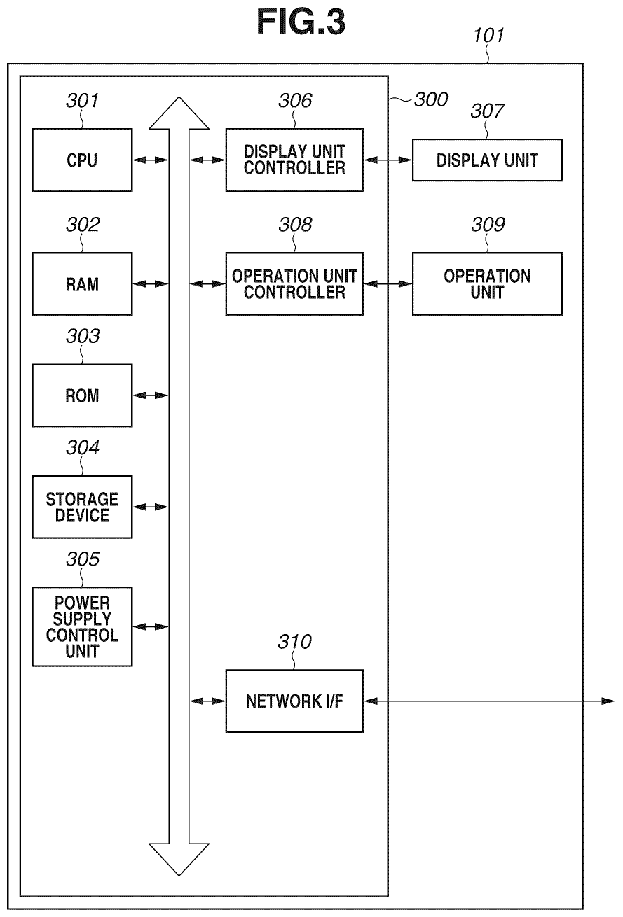

FIG. 3 is a diagram illustrating an example of a hardware configuration of the server 101 illustrated in FIG. 1. The server 101 includes a controller unit 300, a display unit 307, and an operation unit 309.

The controller unit 300 includes a CPU 301, a RAM 302, a ROM 303, a storage device 304, a power supply control unit 305, a display unit controller 306, an operation unit controller 308, and a network I/F 310. The operation unit 309 includes a keyboard, a pointing device, and the like.

The CPU 301 is a central processing unit for controlling the entire server 101.

The RAM 302 is a work memory for the CPU 301 to operate with, and is used for loading various programs therein and storing calculation processing results.

The ROM 303 is a memory for storing a start-up program of the CPU 301 and various kinds of setting information.

The storage device 304 is a nonvolatile memory for storing large size programs and data, and is a flash memory or an HDD in the controller unit 300.

The power supply control unit 305 controls electric power required for the server 101 to operate.

The display unit controller 306 transmits display data to the display unit 307, based on a communication protocol of the display unit 307.

The operation unit controller 308 receives an input from the operation unit 309, and converts the received input into data understandable by the CPU 301. In addition, the operation unit 309 and the display unit 307 may be integrally configured as a touch panel.

The network I/F 310 transmits and receives data to and from the network 104 illustrated in FIG. 1.

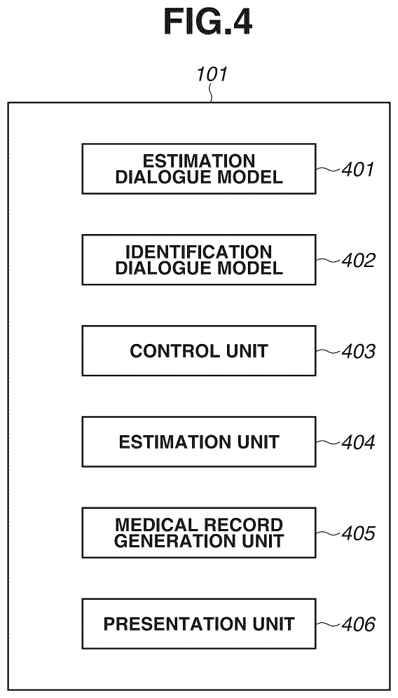

FIG. 4 is a block diagram illustrating an example of a functional configuration of the server 101 illustrated in FIG. 1. The server 101 implements functional configuration units illustrated in FIG. 4 by the CPU 301 illustrated in FIG. 3 executing a program.

The server 101 includes an estimation dialogue model 401, an identification dialogue model 402, a control unit 403, an estimation unit 404, a medical record generation unit 405, and a presentation unit 406.

The estimation dialogue model 401 and the identification dialogue model 402 are, for example, an artificial intelligence (AI) chatbot, which is a program for the AI that has learned by itself based on data and logs to respond to a user's statement, and returns an appropriate response to the user's natural language input.

The estimation dialogue model 401 asks a question to a user, and repeats appropriate questions to the user based on the contents of the user's responses to estimate the user's mental condition. In this case, examples of the mental condition include stress, anxiety, depression, anger, trauma, loss of self-confidence, and loneliness, and the like. The estimated mental condition may be quantitatively expressed in numeric values. Alternatively, the estimated mental condition may be expressed, for example, “stress is high” or “a bit high” in a manner other than the numeric values.

The identification dialogue model 402 is used to ask a question to a user, and repeats appropriate questions to the user based on the contents of the user's responses, to identify the candidates of the user's symptoms.

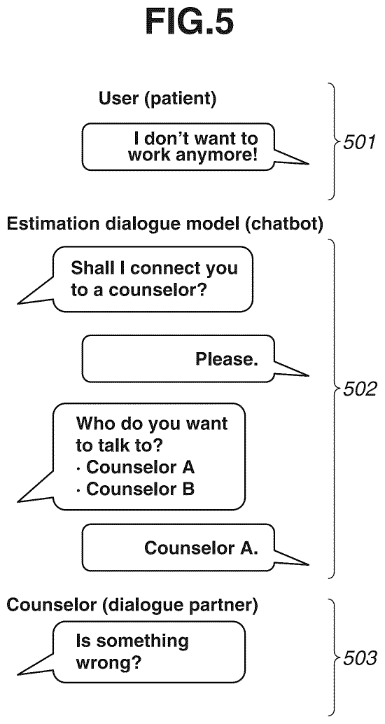

The user can perform the dialogue between the user, the estimation dialogue model 401, and the counselor as illustrated in FIG. 5, by instructing the user counseling application 107 of the user's information terminal 102 to run.

FIG. 5 is a diagram illustrating an example of a screen of dialogues 501 to 503 displayed on the display unit 307 of the user's information terminal 102. In FIG. 5, the user (patient) is a user (client) who is using the user's information terminal 102. The estimation dialogue model (chatbot) 401 is a dialogue model in the server 101. The counselor is a counselor who is using the counselor's information terminal 103.

The dialogue 501 is a statement from the user to the estimation dialogue model 401. The dialogue 502 is a dialogue between the estimation dialogue model 401 and the user. The dialogue 503 is a statement from the counselor to the user.

The estimation dialogue model 401 performs the dialogue 501 with the user. The estimation unit 404 estimates the user's mental condition by analyzing the dialogue 501 between the user and the estimation dialogue model 401.

The estimation unit 404 may also estimate the user's mental condition by analyzing the dialogue 501 between the user and the estimation dialogue model 401 and the information detected by the user's wearable device 109. Examples of the above-described information include information about ambient light, acceleration, a gyroscope, heartbeat, atmospheric pressure, a compass, and blood oxygen level.

For the user, the estimation dialogue model 401 performs the dialogue 502 for changing the dialogue between the user and the estimation dialogue model 401 to a dialogue between the user and the counselor, based on the mental condition estimated by the estimation unit 404.

In the dialogue 502, the estimation dialogue model 401 presents one or more counselor candidates based on the user's information. For example, the estimation dialogue model 401 presents the candidates of counselors A and B. The user states that the user selects the counselor A. The information on the above-described user is, for example, age or address. The estimation dialogue model 401 can present a counselor of a clinic located near the user's address.

In a case where the user approved in the dialogue 502 for changing the dialogue to the dialogue between the user and the counselor, the estimation dialogue model 401 may inquire to the counselor about whether to perform the dialogue between the user and the counselor.

In this case, the estimation dialogue model 401 can present to the counselor a summary of the content of the dialogue 501 between the user and the estimation dialogue model 401.

In a case where the counselor approves the above-described inquiry, the control unit 403 performs control to perform the dialogue 503 between the user and the counselor after the dialogue 502 between the user and the estimation dialogue model 401.

In the dialogue 503, the estimation dialogue model 401 can provide the counselor with a draft response to the user, based on the content of the dialogue 501 between the user and the estimation dialogue model 401, and/or the content of the dialogue 503 between the user and the counselor.

A path 105 in FIG. 1 is a path for the dialogues 501 and 502 between the user and the estimation dialogue model 401. A path 106 in FIG. 1 is a path for the dialogue 503 between the user and the counselor.

The dialogues 501 and 502 between the user and the estimation dialogue model 401 described above are dialogues performed by using characters. The dialogue 503 between the user and the counselor is also a dialogue performed by using characters.

While the dialogues 501 and 502 between the user and the estimation dialogue model 401 are dialogues performed using characters, the dialogue 503 between the user and the counselor may be a dialogue including a voice communication or a video communication.

As illustrated in FIG. 5, the dialogues 501 to 503 are displayed on the same screen.

After the dialogue 503, the medical record generation unit 405 generates a medical record based on the content of the dialogue 501 between the user and the estimation dialogue model 401 and/or the content of the dialogue 503 between the user and the counselor. The medical record is, for example, a medical record in subjective information(S), objective information (O), assessment (A), and plan (P) (SOAP) format. The medical record may include the findings of an analysis result of the above-described voice communication or the video communication. The details thereof will be described below.

The medical record generation unit 405 then performs control so that only the counselor can download the medical record, or presents the medical record only to the counselor.

After the dialogue 503 between the user and the counselor, as a regular follow-up, the presentation unit 406 receives the information detected by the user's wearable device 109, and presents the received information and/or the analysis result of the received information to the counselor. Examples of the above-described information include information about ambient light, acceleration, a gyroscope, heartbeat, atmospheric pressure, a compass, and blood oxygen level.

In the dialogue 501, the identification dialogue model 402 may identify one or more symptom candidates based on the dialogue 501 with the user. In this case, the estimation dialogue model 401 or the identification dialogue model 402 performs a dialogue to change the dialogue to the dialogue between the user and the dialogue partner after identifying the above-described one or more symptom candidates. Before the dialogue 503, the estimation dialogue model 401 or the identification dialogue model 402 presents the identified one or more symptom candidates to the counselor.

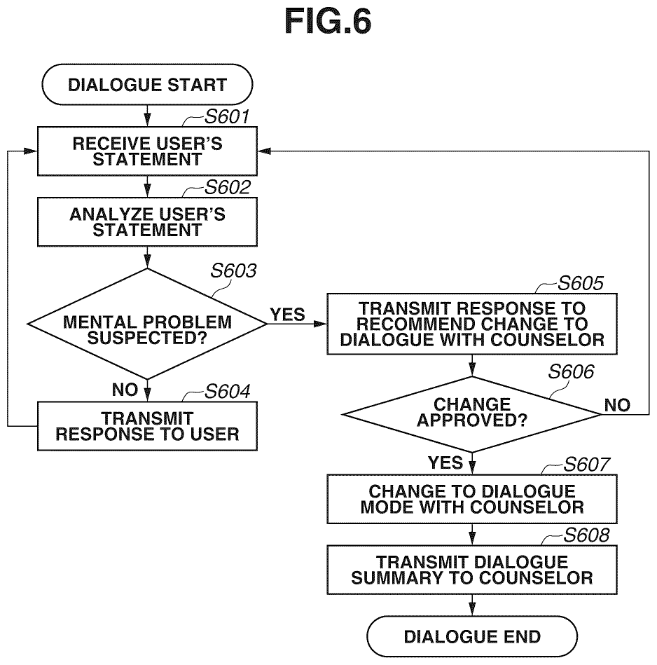

FIG. 6 is a flowchart illustrating a processing method performed by the server 101. When a user instructs the user's information terminal 102 to execute the user counseling application 107, the server 101 starts a dialogue between the user and the estimation dialogue model 401 in FIG. 6.

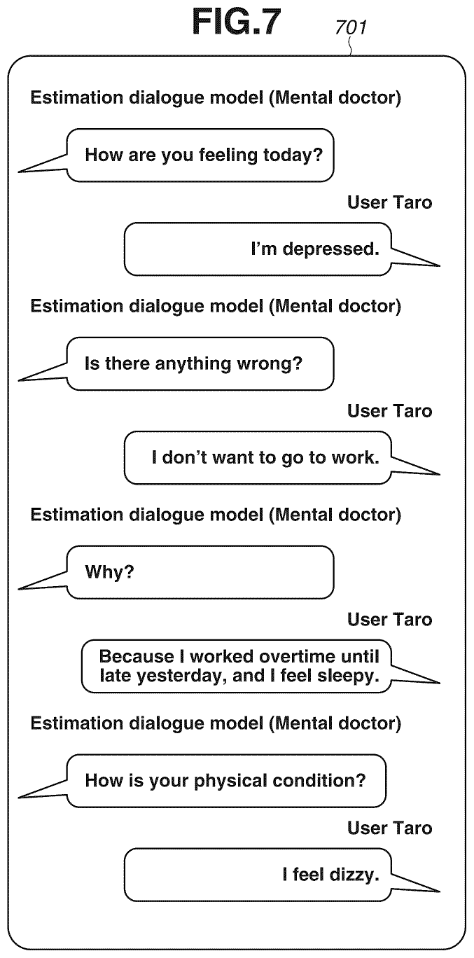

In step S601, the estimation unit 404 performs a dialogue 701 with the user using the estimation dialogue model 401, as illustrated in FIG. 7, and receives the user's statement. FIG. 7 illustrates an example of a screen of the dialogue 701 displayed on the display unit 307 of the user's information terminal 102. The dialogue 701 corresponds to the dialogue 501 in FIG. 5, and is a dialogue between the estimation dialogue model 401 and the user. The user answers questions from the estimation dialogue model 401.

The format of the user's answer is not limited to a free-answer input format, and the user's answer may be selectable from answer candidates. For example, the estimation unit 404 asks the question of “How is your physical condition?” and then can present a plurality of answer candidates (e.g., “I feel dizzy.” or “I feel fatigued.”), using the estimation dialogue model 401.

In step S602, the estimation unit 404 estimates the user's mental condition by analyzing the dialogue 701 between the user and the estimation dialogue model 401 using the estimation dialogue model 401.

The estimation unit 404 may estimate the user's mental condition by analyzing the dialogue 701 between the user and the estimation dialogue model 401, and the information detected by the user's wearable device 109. Examples of the above-described information include information about ambient light, acceleration, a gyroscope, heartbeat, atmospheric pressure, a compass, and blood oxygen level.

The information on the ambient light is used for analyzing the user's sleep pattern or day-and-night rhythm. The acceleration and the gyroscope are used for analyzing the user's activity level or movement pattern. The heartbeat is used for analyzing the user's stress level acquired from heart rate variability.

In step S603, the estimation unit 404 determines whether the user is suspected of having a mental disorder based on the user's mental condition. In a case where the user is not suspected of having a mental disorder (NO in step S603), the processing proceeds to step S604. In step S604, the estimation unit 404 transmits the response to the user. Then, the processing returns to step S601, and the above-described processing is repeated. In a case where the user is suspected of having a mental disorder (YES in step S603), the processing proceeds to step S605.

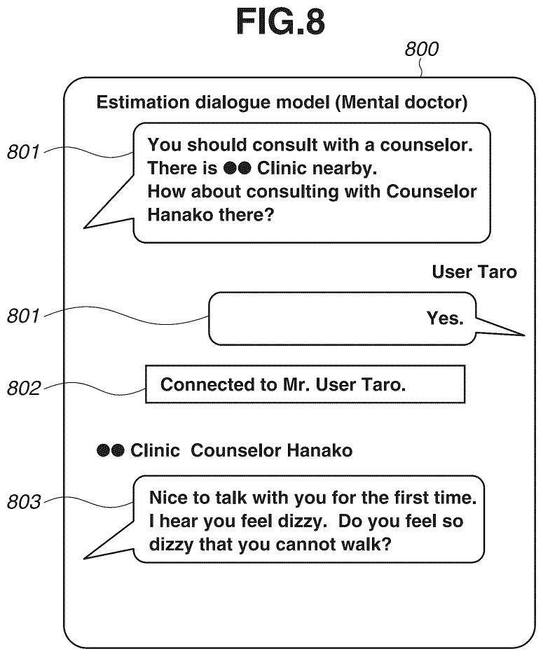

In step S605, the estimation dialogue model 401 performs a dialogue 801 for the user to change the dialogue between the user and the estimation dialogue model 401 to the dialogue between the user and the counselor, as illustrated in FIG. 8. FIG. 8 illustrates an example of a screen 800 of the dialogue displayed on the display unit 307 of the user's information terminal 102. The dialogue 801 corresponds to the dialogue 502 in FIG. 5, and is a dialogue, performed only using characters, between the estimation dialogue model 401 and the user.

In the dialogue 801, the estimation dialogue model 401 presents one or more counselor candidates based on the user's information. The user information is, for example, age or address. The estimation dialogue model 401 can present a counselor of a clinic located near the user's address. In addition, like the dialogue 502 illustrated in FIG. 5, the estimation dialogue model 401 may present a plurality of counselor candidates based on the user information.

In step S606, the estimation dialogue model 401 determines whether the user has approved changing the dialogue to the counselor in the dialogue 801. In the example illustrated in dialogue 801 in FIG. 8, the user approves. In a case where the estimation dialogue model 401 determines that the user has not approved (NO in step S606), the processing returns to step S601. In a case where the estimation dialogue model 401 determines that the user has approved (YES in step S606), the processing proceeds to step S607.

The setting of “change the dialogue automatically” or “confirm whether to approve when changing the dialogue” may be enabled by setting of the user counseling application 107. In the case of “change the dialogue automatically”, the processing skips the processing in steps S605 and S606, and proceeds to step S607.

In step S607, the control unit 403 changes the dialogue mode between the estimation dialogue model 401 and the user to the dialogue mode between the estimation dialogue model 401 and the counselor.

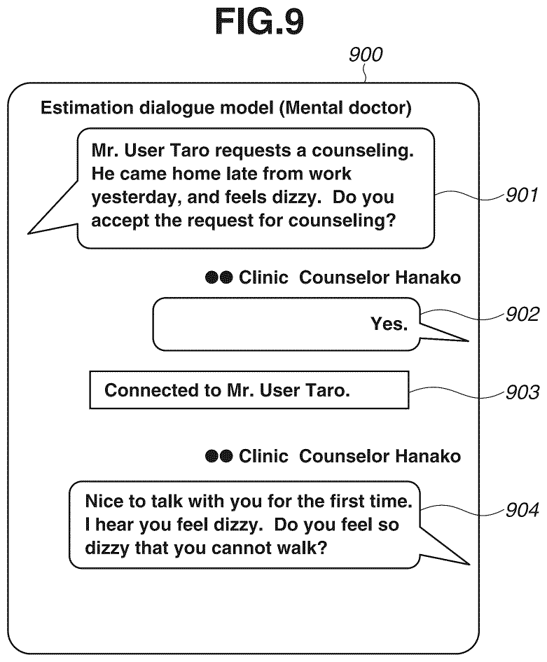

In step S608, the estimation dialogue model 401 presents to the counselor a summary of the content of the dialogue between the user and the estimation dialogue model 401, like a dialogue 901 illustrated in FIG. 9, and inquires of the counselor whether to perform the dialogue between the user and the counselor. FIG. 9 illustrates an example of a screen 900 of the dialogue displayed on the display unit 307 of the counselor's information terminal 103.

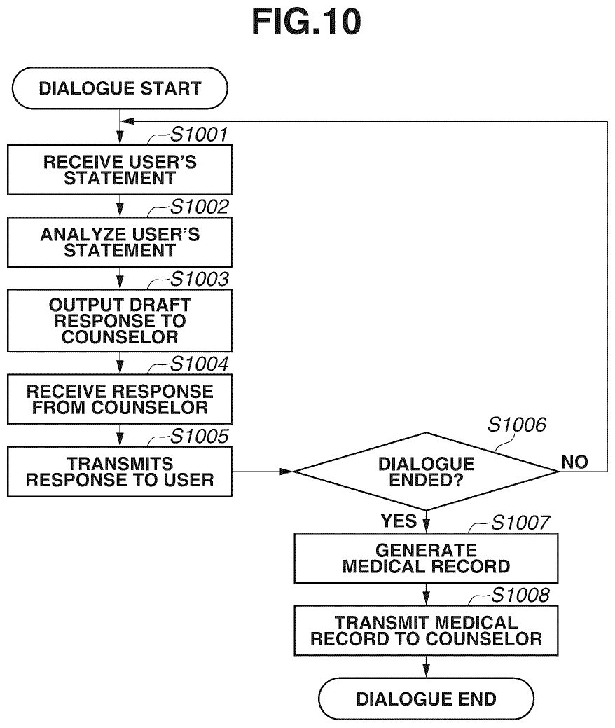

The dialogue 901 and a dialogue 902 are dialogues between the estimation dialogue model 401 and the counselor. In a case where the counselor has approved the above-described inquiry, the estimation dialogue model 401 ends the dialogue mode between the estimation dialogue model 401 and the counselor, and the processing proceeds to the processing in FIG. 10.

FIG. 10 is a flowchart illustrating a processing method performed by the server 101 subsequent to the processing in FIG. 6.

As illustrated in FIGS. 8 and 9, the control unit 403 displays a notification 802 for starting the dialogue mode between the user and the counselor, and a dialogue 803. Then, the control unit 403 performs control to perform the dialogues 803 and 904 between the user and the counselor illustrated in FIGS. 8 and 9. The dialogues 803 and 904 are the statements from the counselor to the user.

On the screen 800 of the user's information terminal 102, the dialogue is continued on the same screen 800, even when the dialogue 801 between the user and the estimation dialogue model 401 is changed to the dialogue 803 between the user and the counselor.

Similarly, on the screen 900 of the counselor's information terminal 103, the dialogue is continued on the same screen 900, even when the dialogues 901 and 902 between the estimation dialogue model 401 and the counselor are changed to the dialogue 904 between the user and the counselor.

Since the dialogue 901, which is a summary of the content of the dialogue between the user and the estimation dialogue model 401, is displayed on the screen 900 of the counselor's information terminal 103, the counselor does not need to ask the same questions again.

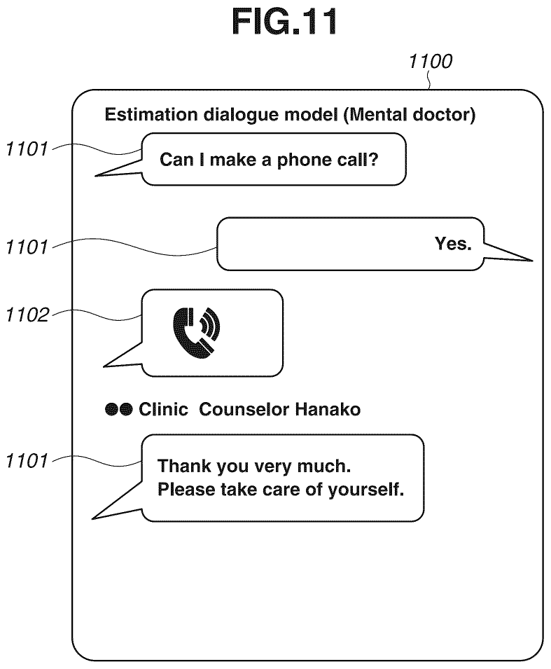

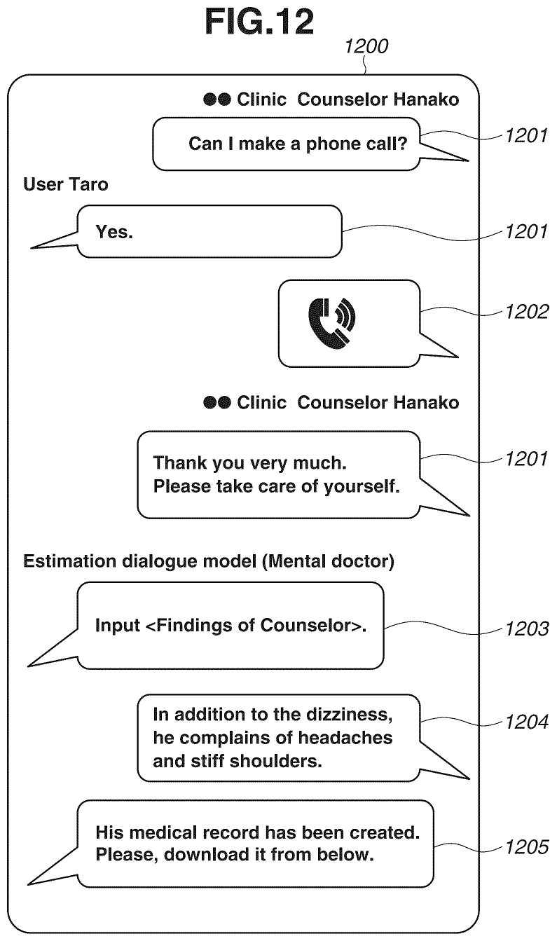

FIG. 11 illustrates an example of a screen 1100 of the dialogue displayed on the display unit 307 of the user's information terminal 102, subsequent to the screen 800 in FIG. 8. FIG. 12 illustrates an example of a screen 1200 of the dialogue displayed on the display unit 307 of the counselor's information terminal 103, subsequent to the screen 900 in FIG. 9.

A dialogue 1101 in FIG. 11 corresponds to a dialogue 1201 in FIG. 12. A dialogue 1102 in FIG. 11 corresponds to a dialogue 1202 in FIG. 12. The dialogues 1101 and 1201 between the user and the counselor are dialogues performed by using characters. The dialogues 1102 and 1202 between the user and the counselor are dialogues performed by using a voice communication or a video communication. In a case where the dialogue becomes the dialogue mode between the user and the counselor, the control unit 403 permits the use of the voice communication and the video communication.

In the user's information terminal 102, the screen subsequent to the screen of the dialogue 701 in FIG. 7 is the screen 800 in FIG. 8, and the screen subsequent to the screen 800 in FIG. 8 is the screen 1100 in FIG. 11. The screen of the dialogue 701, the screen 800, and the screen 1100 are displayed on the same screen.

Similarly, in the counselor's information terminal 103, the screen subsequent to the screen 900 in FIG. 9 is the screen 1200 in FIG. 12. The screen 900 and the screen 1200 are displayed on the same screen.

Returning to FIG. 10, in step S1001, the estimation dialogue model 401 receives the user's dialogue (statement) in FIG. 11.

In step S1002, the estimation dialogue model 401 analyzes the content of the user's dialogue in FIG. 7 and/or the content of the user's dialogue in FIG. 11.

In step S1003, the estimation dialogue model 401 presents (outputs) to the counselor a draft response to the user based on the analysis result in step S1001. The draft response is, for example, a candidate question. The counselor can provide the response with reference to the draft response.

In step S1004, the estimation dialogue model 401 receives the response dialogue from the counselor in FIG. 12.

In step S1005, the estimation dialogue model 401 transmits the response dialogue of the counselor to the user. In this way, the response dialogue of the counselor is displayed on the screen 1100 of the user's information terminal 102 in FIG. 11.

In step S1006, the estimation dialogue model 401 determines whether the dialogue between the user and the counselor has ended. In a case where the estimation dialogue model 401 determines that the dialogue has not ended (NO in step S1006), the processing returns to step S1001. In a case where the estimation dialogue model 401 determines that the dialogue has ended (YES in step S1006), the processing proceeds to step S1007.

In step S1007, the estimation dialogue model 401 transmits to the counselor a dialogue 1203 requesting the input of <counselor's findings>, and receives from the counselor a dialogue 1204 about the content of <counselor's findings>.

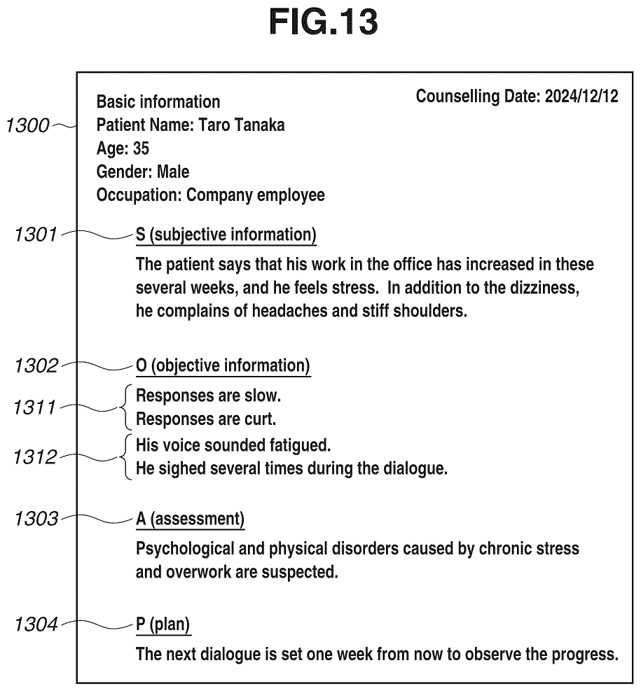

The medical record generation unit 405 generates a medical record 1300 as illustrated in FIG. 13, based on the content of the dialogue between the user and the estimation dialogue model 401, the content of the dialogue between the user and the counselor, and the content of the dialogue between the counselor and the estimation dialogue model 401.

Returning to FIG. 10, in step S1008, the medical record generation unit 405 performs control to enable only the counselor to download the medical record or presents the medical record only to the counselor, and transmits the statement of a dialogue 1205 to the counselor using the estimation dialogue model 401. While the counselor can view the medical record, the user cannot view the medical record.

The control unit 403 then ends the dialogue mode between the counselor and the estimation dialogue model 401.

FIG. 13 illustrates an example of the medical record 1300 generated in step S1007 in FIG. 10. The medical record 1300 is a medical record in a SOAP format including an S (subjective information) 1301, an O (objective information) 1302, an A (assessment) 1303, and a P (plan) 1304.

The S (subjective information) 1301 includes the content of the dialogue between the user and the counselor as well as the content of the dialogue between the user and the estimation dialogue model 401.

The O (objective information) 1302 includes findings 1311 based on the analysis result of the dialogue 1101 performed by using the characters in FIG. 11, and findings 1312 based on the analysis result of the dialogue 1202 performed by the voice communication or the video communication.

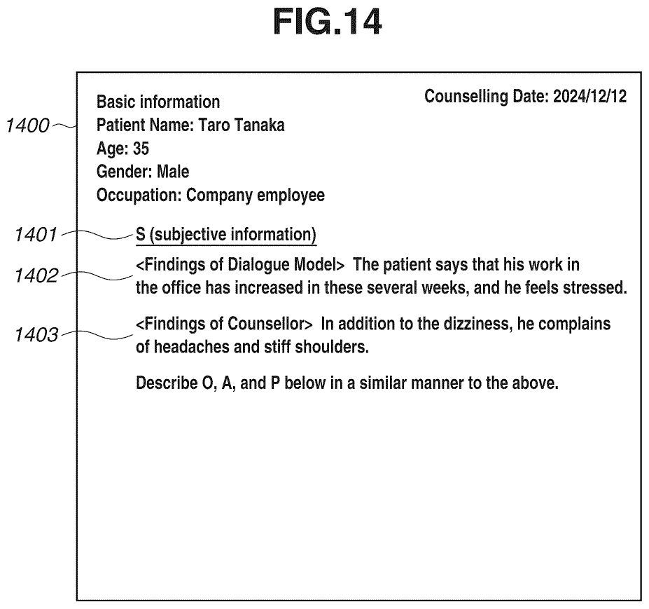

FIG. 14 illustrates an example of another medical record 1400 generated in step S1007 in FIG. 10.

The medical record 1400 is a medical record in the SOAP format, and includes an S (subjective information) 1401. The S (subjective information) 1401 is described separately as findings 1402 by the estimation dialogue model 401 and findings 1403 by the counselor. The findings 1402 of the estimation dialogue model 401 are findings based on the analysis result of the content of the dialogue between the estimation dialogue model 401 and the user. The findings 1403 by the counselor are findings based on the dialogue 1204 between the user and the counselor in FIG. 12.

The O, the A, and the P are also described in a similar manner to the S (subjective information) 1401.

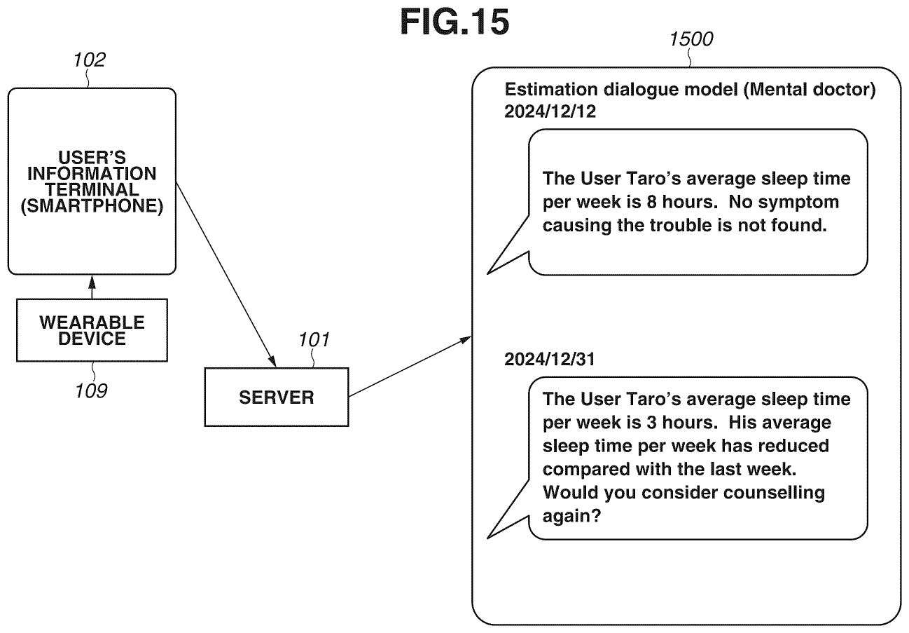

FIG. 15 is a diagram illustrating follow-up processing performed by the server 101 after the processing of the flowchart in FIG. 10.

The user's wearable device 109 detects the information about ambient light, acceleration, a gyroscope, heartbeat, atmospheric pressure, a compass, blood oxygen level, and the like. The user's information terminal 102 periodically receives the information detected by the wearable device 109, and transmits the detected information to the server 101.

The presentation unit 406 of the server 101 receives the information detected by the user's wearable device 109, and presents, using the estimation dialogue model 401, the received information and/or the analysis result of the received information as illustrated in a screen 1500, using the estimation dialogue model 401 to the counselor. The screen 1500 is a screen displayed on the display unit 307 of the counselor's information terminal 103.

The screen 1500 enables the counselor to know the information on the user as the monitoring over time of the follow-up of the user's condition after the dialogue between the user and the counselor.

### Second Embodiment

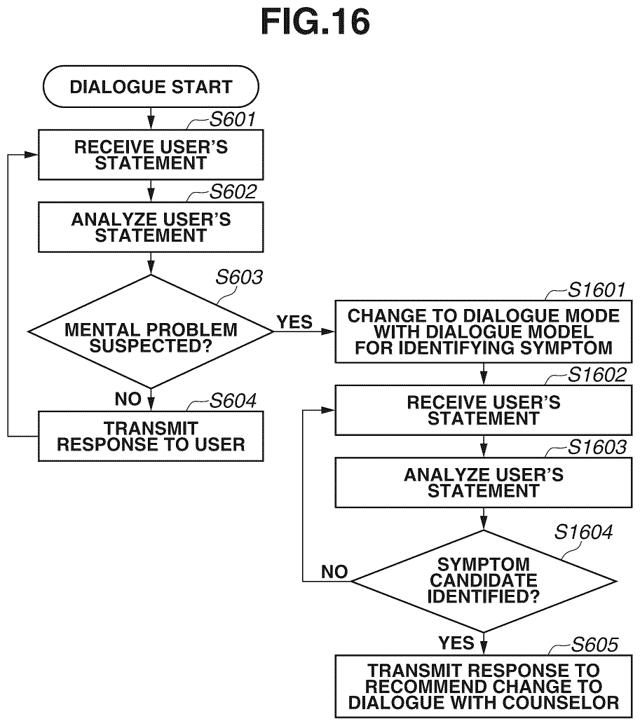

FIG. 16 is a flowchart illustrating a processing method performed by the server 101 according to a second embodiment. FIG. 16 is the flowchart obtained by adding steps S1601 to S1604 to the flowchart in FIG. 6.

Elements of the second embodiment different from the first embodiment will be described below.

In step S603, in the case where the estimation dialogue model 401 determines that the client is suspected of having a mental disorder (YES in step S603), the processing proceeds to step S1601.

In step S1601, the control unit 403 changes the dialogue mode between the user and the estimation dialogue model 401 to the dialogue mode between the user and the identification dialogue model 402.

This change does not require user approval. The identification dialogue model 402 is a dialogue model for identifying a symptom of the user.

In step S1602, the identification dialogue model 402 performs a dialogue (question and answer) between the user and the identification dialogue model 402 for identifying a symptom, and receives a statement (answer) of the user.

In step S1603, the identification dialogue model 402 analyzes the dialogue between the user and the identification dialogue model 402.

In step S1604, the identification dialogue model 402 identifies one or more symptom candidates of the user based on the analysis result in step S1603. Examples of the symptoms include schizophrenia, mood disorder, epilepsy, and the like.

In a case where the reliability of the symptom becomes 70% or more, the identification dialogue model 402 determines that the symptom is identified. The identification dialogue model 402 can identify a plurality of symptoms with a reliability of 70% or more. In a case where one symptom is identified, the possibility of the wrong determination increases. In a case where a plurality of symptoms is identified, the possibility that a correct answer is included therein is high. The final determination of the symptom can be left to the counselor.

The identification dialogue model 402 determines whether one or more symptom candidates are identified in step S1604.

In a case where the identification dialogue model 402 determines that one or more symptom candidates are not identified (NO in step S1604), since the reliability of the symptom is low, the processing returns to step S1602, and asks questions required for identifying the symptom. In a case where the identification dialogue model 402 determines that one or more symptom candidates are identified (YES in step S1604), the processing proceeds to step S605.

The processing in step S605 and subsequent steps is similar to that in FIG. 6. In step S608 in FIG. 6, the estimation dialogue model 401 or the identification dialogue model 402 presents the identified one or more symptom candidates to the counselor. Because the symptom candidates are displayed on the display unit 307 of the counselor's information terminal 103, the counselor can easily perform the final determination of the user's symptom.

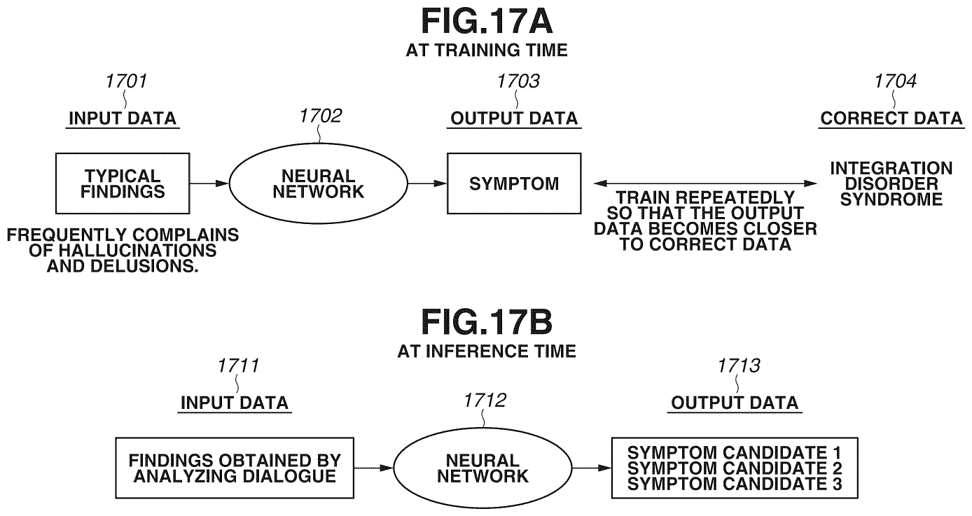

FIG. 17A is a diagram illustrating processing performed by the identification dialogue model 402 during training.

A neural network 1702 is a neural network of the identification dialogue model 402. At the training time, the neural network 1702 receives input data 1701, and outputs output data 1703.

The input data 1701 is typical data on findings, and is data, such as, “The client frequently complains of hallucinations and delusions”. The output data 1703 is a symptom.

The neural network 1702 is repeatedly trained by adjusting parameters so that the output data 1703 comes closer to correct data 1704. The correct data 1704 is correct data (training data) for the input data 1701, and is, for example, “integration disorder syndrome”.

An example of a combination of the input data 1701 and the correct data 1704 in a case where the symptom is the “schizophrenia” will be described. Examples of the input data 1701 include “The client frequently complains of hallucinations and delusions.”, “The client has difficulty organizing thoughts, and the stories jump around.”, “The client has a flattening of emotions, and lacks emotional expressions.”, “The client has few social relationships and tends to avoid socializing.”, and “The client believes the client is special, and often feels scrutinized and attacked by others.”. The correct data 1704 is the “integration disorder syndrome”.

Next, an example of a combination of the input data 1701 and the correct data 1704 in a case where the symptom is “mood disorder (depression)” will be described. Examples of the input data 1701 include “Persistent depressed mood continues and feelings of interest and pleasure have significantly decreased.”, “The client constantly feels fatigued or a lack of energy.”, “The client loses a sense of pride, and feels valueless.”, “Disorder of sleep (excessive sleep or insomnia) is seen.”, and “The client has a change in appetite (excessive appetite or lack of appetite), and a change in weight.”. The correct data 1704 is the “mood disorder (depression)”.

Next, an example of a combination of the input data 1701 and the correct data 1704 in a case where the symptom is “epilepsy” will be described. Examples of the input data 1701 include “A sudden consciousness loss or a convulsive seizure repeatedly occurs.”, “During the seizure, sometimes the body stiffens, and the muscles cramp.”, “After the seizure, the client often feels extremely fatigued and confused.”, “A short-time memory loss sometimes occurs.”, and “A certain stimulus (e.g., light or sound) sometimes triggers the seizure.”. The correct data 1704 is the “epilepsy”.

FIG. 17B is a diagram illustrating processing performed by the identification dialogue model 402 at an inference time.

A neural network 1712 is a neural network of the identification dialogue model 402, and is a trained network obtained by training the neural network 1702 in FIG. 17A. The processing at the inference time corresponds to the processing in step S1604 in FIG. 16. At the inference time, the neural network 1712 receives input data 1711, and outputs output data 1713.

The input data 1711 is data on the findings obtained by analyzing the dialogue between the user and the identification dialogue model 402. The output data 1713 is data on symptom candidates.

The output data 1713 can be a plurality (e.g., three) of symptom candidates. The neural network 1712 outputs a plurality of the symptom candidates, instead of outputting only one symptom, with the reliability of 70% or more. In a case where one symptom is output, the possibility of a wrong symptom increases. In a case where a plurality of symptoms is output, the possibility that a correct answer is included therein is high. The final determination of the symptom can be left to the counselor.

As described above, according to the first and second embodiments, the server 101 can analyze the content of the dialogue between the user and the estimation dialogue model 401, and in the case where the client's mental condition is abnormal, the dialogue between the user and the counselor can be performed without changing the screen. Since a chance for performing the dialogue between the user and the counselor is provided that is easy for the user to use, it is possible to reduce the number of persons who have mental troubles.

The above-described embodiments are merely examples to implement the present disclosure, and are not seen to be limiting. Any implementation that would enable practice of the present disclosure is seen to be applicable.

According to the present disclosure, it is possible to perform a dialogue between a client and a dialogue partner (counselor) based on a mental condition of a client.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2024-221769, filed Dec. 18, 2024, which is hereby incorporated by reference herein in its entirety.

What is claimed is:

1. An information processing apparatus comprising:
a memory storing at least one program; and
at least one processor, that when executing the program, is caused to:
perform a first dialogue with a client; and
estimate a mental condition of the client by analyzing the first dialogue;
wherein, based on the estimated mental condition, perform a dialogue with the client for changing the first dialogue to a second dialogue between the client and a dialogue partner.

2. The information processing apparatus according to claim 1, wherein the at least one processor is further caused to, based on information about the client, present one or more candidates of dialogue partners.

3. The information processing apparatus according to claim 2, wherein the information on the client is at least an age of the client or an address of the client.

4. The information processing apparatus according to claim 1, wherein the first dialogue is a dialogue performed using characters.

5. The information processing apparatus according to claim 1, wherein the at least one processor is further caused to perform the second dialogue after the first dialogue.

6. The information processing apparatus according to claim 1,
wherein the first dialogue and the second dialogue are performed using characters.

7. The information processing apparatus according to claim 1,
wherein the first dialogue is performed using only characters, and
wherein the second dialogue includes using a voice communication or a video communication.

8. The information processing apparatus according to claim 1, wherein the first dialogue and the dialogue to change the first to the second dialogue are performed on a display screen.

9. The information processing apparatus according to claim 1, wherein the first dialogue, the dialogue to change the first to the second dialogue, and the second dialogue are performed on a same display screen.

10. The information processing apparatus according to claim 1, wherein the at least one processor is further caused to generate, after the second dialogue, a medical record based on a content of the first dialogue or a content of the second dialogue.

11. The information processing apparatus according to claim 10, wherein the medical record is a medical record in a subjective, objective, assessment, and plan (SOAP) format.

12. The information processing apparatus according to claim 10,
wherein the second dialogue includes a dialogue performed using a voice communication or a video communication, and
wherein the medical record includes information based on an analysis result of the voice communication or the video communication.

13. The information processing apparatus according to claim 10, wherein the at least one processor is further caused to control the information processing apparatus to enter a state where only the dialogue partner can download the medical record or be provided the medical record.

14. The information processing apparatus according to claim 1, wherein the at least one processor is further caused to estimate the mental condition of the client by analyzing the first dialogue and information detected by a device of the client.

15. The information processing apparatus according to claim 1, wherein the at least one processor is further caused to, after the second dialogue, receive information detected by a device of the client and to present the received information and/or an analysis result of the received information to the dialogue partner.

16. The information processing apparatus according to claim 1,
wherein the at least one processor is further caused to inquire to the dialogue partner whether to perform the second dialogue in a case where the client approves in the dialogue to change the first dialogue to the second dialogue, and to perform the second dialogue in a case where the dialogue partner approves the inquiry.

17. The information processing apparatus according to claim 1, wherein the at least one processor is further caused to present to the dialogue partner a summary of a content of the first dialogue.

18. The information processing apparatus according to claim 1, wherein the at least one processor is further caused to present the dialogue partner with a draft response to the client based on a content of the first dialogue or a content of the second dialogue.

19. A processing method of an information processing apparatus, comprising:
performing a first dialogue with a client;
estimating a mental condition of the client by analyzing the first dialogue; and
performing, based on the estimated mental condition, a dialogue with the client for changing the first dialogue to a second dialogue between the client and a dialogue partner.

20. A non-transitory computer-readable storage medium storing a program for causing a computer to execute the processing method of the information processing apparatus according to claim 19.
